(19) United States
(12) Patent Application Publication
(10) Pub. No.: US 2010/0234453 A1
(43) Pub. Date: Sep. 16, 2010

(54) PHARMACEUTICAL COMPOSITION CONTAINING PHENANTHRENEQUINONE-BASED COMPOUND FOR INTESTINE DELIVERY SYSTEM

(76) Inventors: In Geun Jo, Chungcheongnam-do (KR); Sang-Ku Yoo, Gyeonggi-do (KR); Myung-gyu Park, Gyeonggi-do (KR); Taehwan Kwak, Gyeonggi-do (KR)

Correspondence Address: BIRCH STEWART KOLASCH & BIRCH, PO BOX 747, FALLS CHURCH, VA, 22040-0747, US

(21) Appl. No.: 12/515,013

(22) PCT Filed: Nov. 26, 2007
(86) PCT No.: PCT/KR07/06010

§ 371 Date: May 15, 2009

(30) Foreign Application Priority Data
Nov. 7, 2006 (KR) 10-2006-0117685
Oct. 11, 2007 (KR) 10-2007-0102478

(51) Int. Cl. A61K 31/352; C07D 307/77; C07D 311/78; A61K 31/343

(52) U.S. Cl. 514/453; 549/457; 549/384; 514/468

(57) ABSTRACT

Provided is an oral pharmaceutical composition with improved bioavailability and pharmacokinetic properties of a drug, by increasing a bioabsorption rate and an in vivo retention time of an active ingredient via intestine-targeted formulation of a particular phenanthrenequinone-based compound, or a pharmaceutically acceptable salt, prodrug, solvate or isomer thereof, as an active ingredient.

### FIELD OF THE INVENTION

The present invention relates to an intestine-targeted pharmaceutical composition comprising a Phenanthrenequinone-based compound. More specifically, the present invention relates to an oral pharmaceutical composition with formulation of an intestinal delivery system of a certain Phenanthrenequinone-based compound or a pharmaceutically acceptable salt, prodrug, solvate or isomer thereof, as an active ingredient.

### BACKGROUND OF THE INVENTION

With recent study of the present applicant, it was revealed that, as compounds of similar series to phenanthrenequinone-based compound in accordance with the present invention, naphthoquinone-based compound such as β-lapachone {7,8-dihydro-2,2-dimethyl-2H-naphtho(2,3-b)dihydropyran-7,8-dione}, dunnione {2,3,3-tirmethyl-2,3,4,5-tetrahydro-naphtho(2,3-b) dihydrofuran-6,7-dione}, α-dunnione {2,3,3-tirmethyl-2,3,4,5-tetrahydro-naphtho(2,3-b)dihydrofuran-6,7-dione}, nocardinone A, nocardinone B, lantalucratin A, lantalucratin B, lantalucratin C and the like is effective for prevention or treatment of obesity, diabetic, metabolic diseases, degenerative diseases, and mitochondrial dysfunction-related diseases (Korean Patent Application Nos. 2004-0116339 and 2006-14541).

However, the aforesaid naphthoquinone-based compound is a sparingly-soluble material which is soluble at a low degree of about 2 to 10% only in high-solubility solvents, such as CH₂Cl₂, CHCl₃, CH₂ClCH₂Cl, CH₃CCl₃, Monoglyme, and Diglyme, but is poorly soluble in other ordinary polar or nonpolar solvents. For this reason, the aforesaid naphthoquinone-based compound suffers from various difficulties associated with formulation of preparations for in vivo administration, in spite of excellent pharmacological effects.

Under current circumstances, the aforementioned highly-insoluble naphthoquinone-based compound has a disadvantage of a significant limit in formulation of the compound into desired pharmaceutical preparations. Even though physiological activity of the naphthoquinone-based compound is elucidated by the present applicant, a dosage form of the naphthoquinone-based compound is limited to a formulation for in vivo administration via intravenous injection.

However, when the naphthoquinone-based compound which is a sparingly-soluble drug is administered by itself or in the form of a conventional simple formulation via an oral route, there is substantially no absorption of the compound into the body, that is, the bioavailability of the drug is very low, so it is impossible to exert the intrinsic efficacy of the drug.

Meanwhile, the present applicant has proposed a novel phenanthrenequinone-based compound having the structure of the naphthoquinone-based compound (Korean Patent Application Nos. 2007-0040673). However, the phenanthrenequinone-based compound has also sparingly-soluble problems.

The drugs can exert therapeutic effects only when an active ingredient is absorbed into the body in an amount exceeding a certain concentration; however, a variety of factors are implicated in bioavailability, the degree to which a drug or other substance becomes available to the target tissue after administration. Low bioavailability of the drug or substance raises serious problems in development of drug compositions.

Therefore, in order to sufficiently and satisfactorily exploit inherent pharmacological properties of the phenanthrenequinone-based compounds, there is an urgent need for development and introduction of a method which is capable of maximizing the bioavailability of these drugs.

### SUMMARY OF THE INVENTION

Therefore, the present invention has been made to solve the above problems and other technical problems that have yet to be resolved.

As a result of a variety of extensive and intensive studies and experiments to solve the problems as described above, the inventors of the present invention have discovered that when a sparingly-soluble phenanthrenequinone-based compound is formulated into an intestine-targeted pharmaceutical composition, it is possible to minimize inactivation of the active ingredient which may occur due to internal bodily environment such as stomach, it is possible to solve a problem of low bioavailability suffered by conventional oral administration, and finally it is possible to significantly improve pharmacokinetic properties of the phenanthrenequinone-based compound. The present invention has been completed based on these findings.

In accordance with an aspect of the present invention, the above and other objects can be accomplished by the provision of an oral pharmaceutical composition wherein a phenanthrenequinone-based compound represented by Formula 1 below, or a pharmaceutically acceptable salt, prodrug, solvate or isomer thereof, as an active ingredient, is prepared into an intestine-targeted formulation:

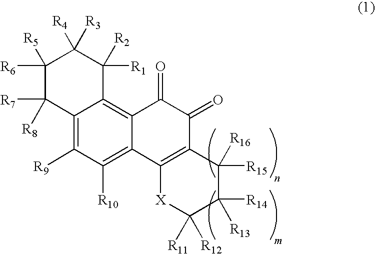

wherein

R₁, R₂, R₃, R₄, R₅, R₆, R₇, R₈, R₉, R₁₀, R₁₁, R₁₂, R₁₃, R₁₄, R₁₅ and R₁₆ are each independently hydrogen, halogen, hydroxyl or C₁-C₆ alkyl, alkene or alkoxy, C₄-C₁₀ cycloalkyl, heterocycloallcyl, aryl or heteroaryl, or two substituents thereof may be taken together to form a cyclic structure or form a double bond;

X is selected from the group consisting of C(R)(R′), N(R″), O and S, wherein R, R′ and R″ are each independently hydrogen or C₁-C₆ lower alkyl; and

m and n each independently are 0 or 1, with proviso that when morn is 0, carbon atoms adjacent to morn form a cyclic structure via a direct bond.

As used in the present specification, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound (I) with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, trichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as arginine, lysine and guanidine, salts with organic bases such as dicyclohexylamine, N-methyl-D-glucamine, tris(hydroxymethyl)methylamine, diethanolamine, choline and triethylamine. The compound in accordance with the present invention may be converted into salts thereof, by conventional methods well-known in the art.

As used herein, the term “prodrug” means an agent that is converted into the parent drug in vivo. Prodrugs are often useful because, in some situations, they may be easier to administer than the parent drug. They may, for instance, be bioavailable by oral administration, whereas the parent may be not. The prodrugs may also have improved solubility in pharmaceutical compositions over the parent drug. An example of a prodrug, without limitation, would be a compound of the present invention which is administered as an ester (“prodrug”) to facilitate transport across a cell membrane where water-solubility is detrimental to mobility, but which then is metabolically hydrolyzed to the carboxylic acid, the active entity, once inside the cell where water solubility is beneficial. A further example of the prodrug might be a short peptide (polyamino acid) bonded to an acidic group, where the peptide is metabolized to reveal the active moiety.

As an example of such prodrug, the pharmaceutical compounds in accordance with the present invention can include a prodrug represented by Formula Ia below as an active material:

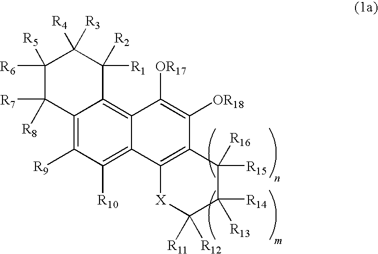

wherein,

R₁, R₂, R₃, R₄, R₅, R₆, R₇, R₈, R₉, R₁₀, R₁₁, R₁₂, R₁₃, R₁₄, R₁₅, R₁₆, m, n and X are as defined in Formula 1.

R₁₇ and R₁₈ are each independently —SO₃—Na⁺ or substituent represented by Formula 2 below or a salt thereof,

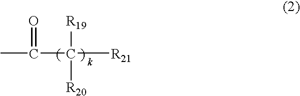

wherein,
R₁₉ and R₂₀ are each independently hydrogen or substituted or unsubstituted C₁˜C₂₀ linear alkyl or C₁˜C₂₀ branched alkyl
R₂₁ is selected from the group consisting of substituents i) to viii) below:
i) hydrogen;
ii) substituted or unsubstituted C₁˜C₂₀ linear alkyl or C₁˜C₂₀ branched alkyl;
iii) substituted or unsubstituted amine;
iv) substituted or unsubstituted C₃˜C₁₀ cycloalkyl or C₃˜C₁₀ heterocycloalkyl;
v) substituted or unsubstituted C₄˜C₁₀ aryl or C₄˜C₁₀ heteroaryl;
vi) —(CRR′—NR″CO)₁—R₂₂, wherein R, R′ and R″ are each independently hydrogen or substituted or unsubstituted C₁˜C₂₀ linear alkyl or C₁˜C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃—Na⁺;
k is selected from the 0˜20, with proviso that when k is 0, R₁₉ and R₂₀ are not anything, and R₂₁ is directly bond to a carbonyl group.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof, that has the same chemical formula or molecular formula but is optically or sterically different therefrom. D type optical isomer and L type optical isomer can be present in the Formula 1, depending on the R₁˜R₁₆ types of substituents selected.

Unless otherwise specified, the term “phenanthrenequinone-based compound” is intended to encompass a compound per se, and a pharmaceutically acceptable salt, prodrug, solvate and isomer thereof.

As used herein, the term “alkyl” refers to an aliphatic hydrocarbon group. The alkyl moiety may be a “saturated alkyl” group, which means that it does not contain any alkene or alkyne moieties. Alternatively, the alkyl moiety may also be an “unsaturated alkyl” moiety, which means that it contains at least one alkene or alkyne moiety. The term “alkene” moiety refers to a group in which at least two carbon atoms form at least one carbon-carbon double bond, and an “alkyne” moiety refers to a group in which at least two carbon atoms form at least one carbon-carbon triple bond. The alkyl moiety, regardless of whether it is substituted or unsubstituted, may be branched, linear or cyclic.

As used herein, the term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

As used herein, the term “aryl” refers to an aromatic substituent group which has at least one ring having a conjugated pi (π) electron system and includes both carbocyclic aryl (for example, phenyl) and heterocyclic aryl (for example, pyridine) groups. This term includes monocyclic or fused-ring polycyclic (i.e., rings which share adjacent pairs of carbon atoms) groups.

As used herein, the term “heteroaryl” refers to an aromatic group that contains at least one heterocyclic ring.

Examples of aryl or heteroaryl include, but are not limited to, phenyl, furan, pyran, pyrimidyl and triazyl.

R₁, R₂, R₃, R₄, R₅, R₆, R₇, R₈, R₉, R₁₀, R₁₁, R₁₂, R₁₃, R₁₄, R₁₅ and R₁₆ in Formula 1 in accordance with the present invention may be optionally substituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino including mono and di substituted amino, and protected derivatives thereof. Further, in Formula 2, where R₁₉, R₂₀ and R₂₀ are substituted, they may be substituted by the above substituents.

Among compounds of Formula 1 in accordance with the present invention, preferred are compounds of Formulas 3 and 6 below.

Compounds of Formula 3 below are compounds wherein m is 1, n is 0 and adjacent carbon atoms form a cyclic structure (furan ring) via a direct bond therebetween and are often referred to as ‘furanotetrahydrophenanthrene compounds’ or ‘furanotetrahydro-3,4-phenanthrenequinone’ hereinafter.

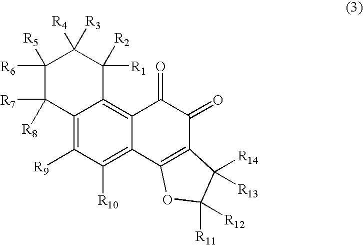

Compounds of formula 4 below are compounds wherein m and n is respectively 1 and are often referred to as ‘pyranotetrahydrophenanthrene compounds’ or ‘pyranotetrahydro-3,4-phenanthrenequinone’ hereinafter.

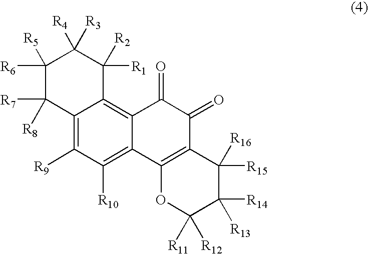

In the aforesaid pyranotetrahydrophenanthrene compounds and pyranotetrahydro-3,4-phenanthrenequinone, it is also possible that R₂ and R₄ and/or R₆ and R₈ form a chemical bond. In this regard, when m and n are respectively 0 and 1, the compounds are classified into two types of formula 5 and formula 6 below.

That is, compounds of Formula 5, wherein m is 1, n is 0 and adjacent carbon atoms form a cyclic structure (furan ring) via a direct bond therebetween, are often referred to as ‘furanophenanthrene compounds’ or ‘furan-3,4-phenanthrenequinone’ hereinafter.

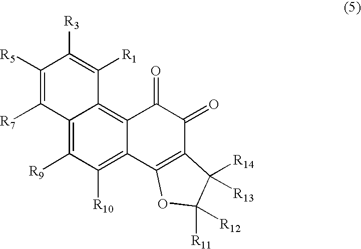

Compounds of Formula 6, wherein m and n are respectively 1, are often referred to as ‘pyranophenanthrene compounds’ or ‘pyrano-3,4-phenanthrenequinone’ hereinafter.

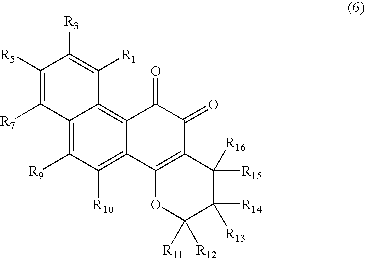

The term “pharmaceutical composition” as used herein means a mixture of a compound of Formula 1 as an active material and other components which are required for an intestine-targeted formulation.

### Preparation of Active Materials

In the pharmaceutical composition in accordance with the present invention, compounds of Formula 1 which are active materials, as will be illustrated hereinafter, can be prepared. The preparation processes described below are only exemplary ones and other processes can also be employed. As such, the scope of the instant invention is not limited to the following processes.

In general, tricyclic naphthoquinone (pyrano-o-naphthoquinone and furano-o-naphthoquinone) derivatives can be synthesized mainly by two methods. One is to derive cyclization reaction using 3-allyl-2-hydroxy-1,4-naphthoquinone in acid catalyst condition, like in the following β-lapachone synthesis method. In the present invention, when a compound in which R₁₁ and R₁₂ are not hydrogen simultaneously, most of compounds of formula 1 were synthesized on the basis of that method.

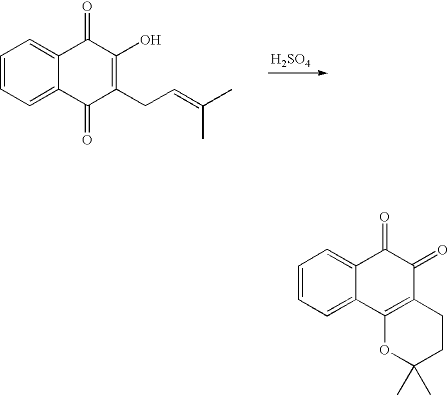

That is, 3-allyloxy-1,4-phenanthrenequinone can be obtained by deriving Diels-Alder reaction between 2-allyloxy-1,4-benzoquinone and styrene or 1-vinylcyclohexane derivatives and dehydrating the resulting intermediates using oxygen present in the air or oxidants such as NaIO₄ and DDQ. By further re-heating the above compound, 2-allyl-3-hydroxy-1,4-phenanthrenequinone of Lapachole form can be synthesized via Claisen rearrangement.

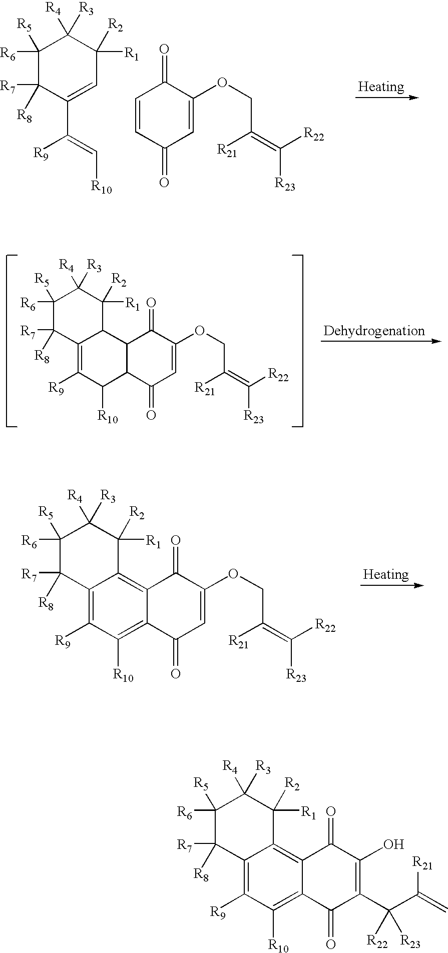

When the thus obtained 2-allyl-3-hydroxy-1,4-phenanthrenequinone is ultimately subject to cyclization in an acid catalyst condition, various 3,4-phenanthrenequinone-based or 5,6,7,8-tetrahydro-3,4-phenanthrenequinone-based compounds can be synthesized. In this case, 5 or 6-cyclic cyclization occurs depending on the types of substituents (R₂₁, R₂₂, R₂₃ in the above formula) represented in the above formula, and also they are converted to the corresponding, adequate substituents (R₁₁, R₁₂, R₁₃, R₁₄, R₁₅, R₁₆).

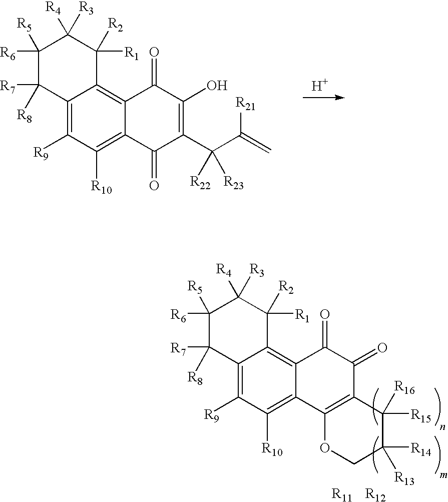

Further, 3-allyloxy-1,4-phenanthrenequinone is hydrolyzed to 3-oxy-1,4-phenanthrenequinone, in the condition of acid or alkali (OH) catalyst, which is then reacted with various allyl halides to synthesize 2-allyl-3-hydroxy-1,4-phenanthrenequinone by C-alkylation. The thus obtained 2-allyl-3-hydroxy-1,4-phenanthrenequinone derivatives are subject to cyclization in the condition of acid catalyst to synthesize various 3,4-phenanthrenequinone-based or 5,6,7,8-tetrahydro-3,4-naphthoquinone-based compounds. In this case, 5 or 6-cyclic cyclization occurs depending on the types of substituents (R₂₁, R₂₂, R₂₃, R₂₄ in the above formula) represented in the above formula, and also they are converted to the corresponding, adequate substituents (R₁₁, R₁₂, R₁₃, R₁₄, R₁₅, R₁₆).

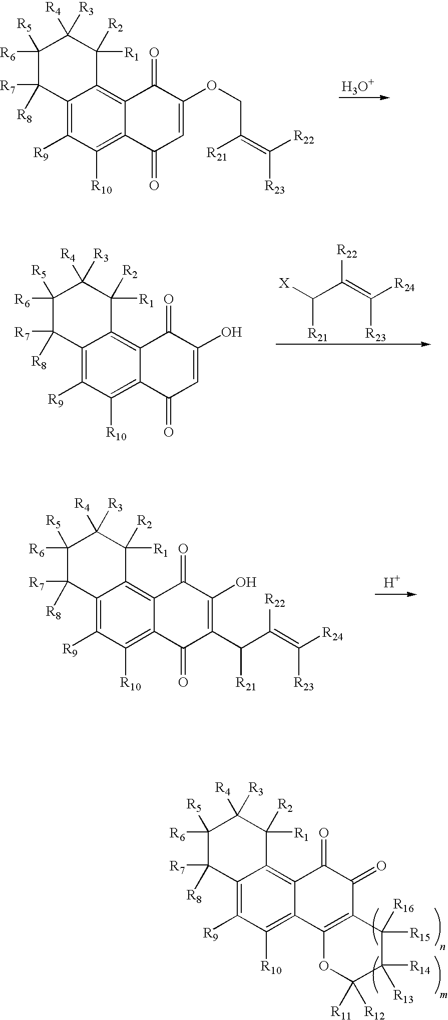

However, compounds in which substituents R₁₁ and R₁₂ are hydrogen simultaneously cannot be obtained by cyclization in the condition of acid catalyst. These compound were obtained on the basis of the method reported by J. K. Snyder (Tetrahedron Letters, 28, 3427˜3430, 1987; Journal of Organic Chemistry, 55, 4995˜5008, 1990), more specifically, by first obtaining furanobenzoquinone, to which a furan ring is introduced, by cyclization, and then obtaining tricyclic phenanthroquinone by cyclization with 1-vinylcyclohexene derivative, followed by reduction via hydrogen addition. The above synthesis process can be summarized as follows.

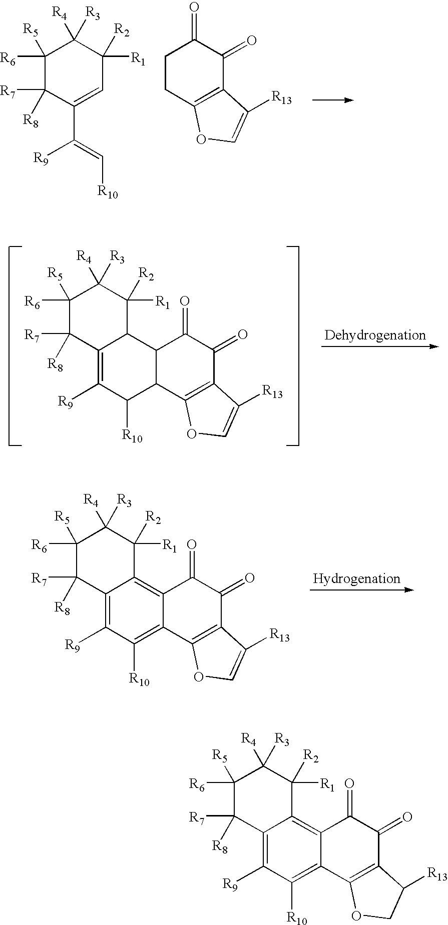

Based on the above-mentioned preparation methods, various derivatives may be synthesized using relevant synthesis methods, depending upon kinds of substituents.

Among compounds of Formula 1 in accordance with the present invention, particularly preferred are exemplified in Table 1 below, but are not limited to, Specific preparation methods will be described in the following Examples.

TABLE 1

| | | | Molecular |
|---|---|---|---|
| No. | Chemical structure | Formula | weight |
| 1 | 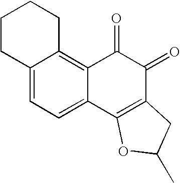 | C₁₇H₁₆O₃ | 268.31 |
| 2 | 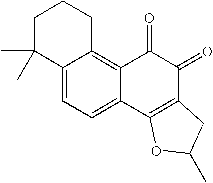 | C₁₉H₂₀O₃ | 296.36 |
| 3 | 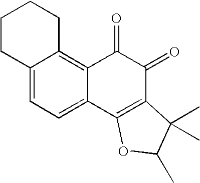 | C₁₉H₂₀O₃ | 296.36 |
| 4 | 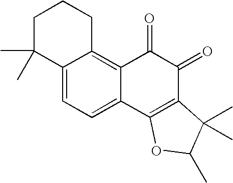 | C₂₁H₂₄O₃ | 324.41 |
| 5 | 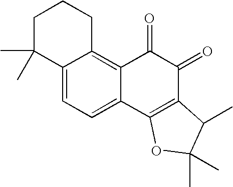 | C₂₁H₂₄O₃ | 324.41 |
| 6 | 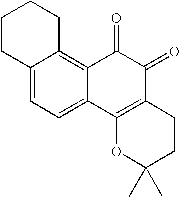 | C₁₉H₂₀O₃ | 296.36 |
| 7 | 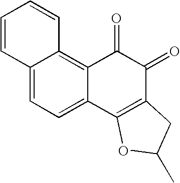 | C₁₇H₁₂O₃ | 264.28 |
| 8 | 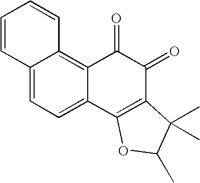 | C₁₉H₁₆O₃ | 292.33 |
| 9 | 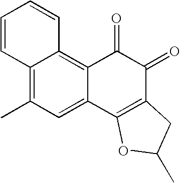 | C₁₈H₁₄O₃ | 278.30 |
| 10 | 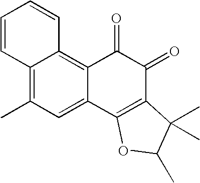 | C₂₀H₁₈O₃ | 306.36 |
| 11 | 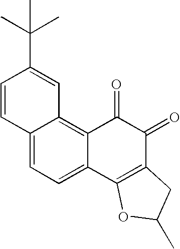 | C₂₁H₂₀O₃ | 320.38 |
| 12 | 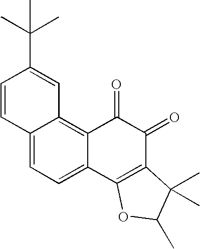 | C₂₃H₂₄O₃ | 348.43 |
| 13 | 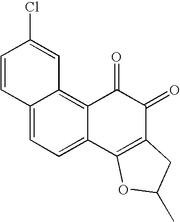 | C₁₇H₁₁ClO₃ | 298.72 |
| 14 | 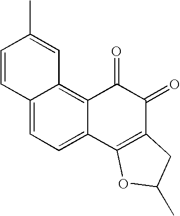 | C₁₈H₁₄O₃ | 278.30 |
| 15 | 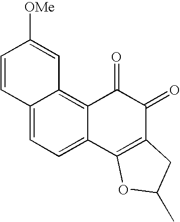 | C₁₈H₁₄O₄ | 294.30 |
| 16 | 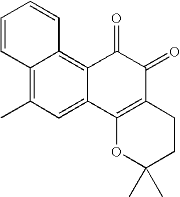 | C₂₀H₁₈O₃ | 306.36 |
| 17 | 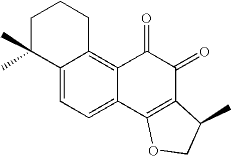 | C₁₈H₁₈O₃ | 282.33 |
| 18 | 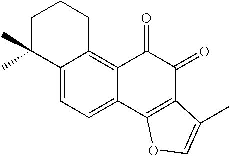 | C₁₈H₁₆O₃ | 280.33 |
| 19 | 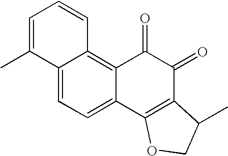 | C₁₈H₁₄O₃ | 278.33 |
| 20 | 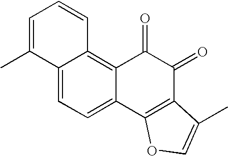 | C₁₈H₁₂O₃ | 276.33 |

Generally, an oral pharmaceutical composition passes through the stomach upon oral administration, is largely absorbed by the small intestine and then diffused into all the tissues of the body, thereby exerting therapeutic effects on the target tissues.

In this connection, the oral pharmaceutical composition according to the present invention enhances bioabsorption and bioavailability of a certain phenanthrenequinone-based compound active ingredient via intestine-targeted formulation of the active ingredient. More specifically, when the active ingredient in the pharmaceutical composition according to the present invention is primarily absorbed in the stomach, and upper parts of the small intestine, the active ingredient absorbed into the body directly undergoes liver metabolism which is then accompanied by substantial degradation of the active ingredient, so it is impossible to exert a desired level of therapeutic effects. On the other hand, it is expected that when the active ingredient is largely absorbed around and downstream of the lower small intestine, the absorbed active ingredient migrates via lymph vessels to the target tissues to thereby exert high therapeutic effects.

Further, as it is constructed in such a way that the pharmaceutical composition according to the present invention targets up to the colon which is a final destination of the digestion process, it is possible to increase the in vivo retention time of the drug and it is also possible to minimize decomposition of the drug which may take place due to the body metabolism upon administration of the drug into the body. As a result, it is possible to improve pharmacokinetic properties of the drug, to significantly lower a critical effective dose of the active ingredient necessary for the treatment of the disease, and to obtain desired therapeutic effects even with administration of a trace amount of the active ingredient. Further, in the oral pharmaceutical composition, it is also possible to minimize the absorption variation of the drug by reducing the between- and within-individual variation of the bioavailability which may result from intragastric pH changes and dietary uptake patterns.

Therefore, the intestine-targeted formulation according to the present invention is configured such that the active ingredient is largely absorbed in the small and large intestines, more preferably in the jejunum, and the ileum and colon corresponding to the lower small intestine, particularly preferably in the ileum or colon.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or higher, as compared to that of the stomach. Based on this fact, the pH-sensitive polymer may be used in order to ensure that the pharmaceutical composition reaches the lower intestinal parts without being affected by pH fluctuations of the digestive tact. Examples of the pH-sensitive polymer may include, but are not limited to, at least one selected from the group consisting of methacrylic acid-ethyl acrylate copolymer (Eudragit: Registered Trademark of Rohm Pharma GmbH), hydroxypropylmethyl cellulose phthalate (HPMCP) and a mixture thereof.

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylase, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

In the oral pharmaceutical composition according to the present invention, the active ingredient may have a crystalline structure with a high degree of crystallinity, or a crystalline structure with a low degree of crystallinity.

As used herein, the term “degree of crystallinity” is defined as the weight fraction of the crystalline portion of the total compound and may be determined by a conventional method known in the art. For example, measurement of the degree of crystallinity may be carried out by a density method or precipitation method which calculates the crystallinity degree by previous assumption of a preset value obtained by addition and/or reduction of appropriate values to/from each density of the crystalline portion and the amorphous portion, a method involving measurement of the heat of fusion, an X-ray method in which the crystallinity degree is calculated by separation of the crystalline diffraction fraction and the noncrystalline diffraction fraction from X-ray diffraction intensity distribution upon X-ray diffraction analysis, or an infrared method which calculates the crystallinity degree from a peak of the width between crystalline bands of the infrared absorption spectrum.

In the oral pharmaceutical composition according to the present invention, the crystallinity degree of the active ingredient is preferably 50% or less. More preferably, the active ingredient may have an amorphous structure from which the intrinsic crystallinity of the material was completely lost. The amorphous phenanthrenequinone-based compound exhibits a relatively high solubility, as compared to the crystalline phenanthrenequinone-based compound, and can significantly improve a dissolution rate and in vivo absorption rate of the drug.

In one preferred embodiment of the present invention, the amorphous structure may be formed during preparation of the active ingredient into microparticles or fine particles (micronization of the active ingredient). The microparticles may be prepared, for example by spray drying of active ingredients, melting methods involving formation of melts of active ingredients with polymers, co-precipitation involving formation of co-precipitates of active ingredients with polymers after dissolution of active ingredients in solvents, inclusion body formation, solvent volatilization, and the like. Preferred is spray drying. Even when the active ingredient is not of an amorphous structure, that is has a crystalline structure or semi-crystalline structure, micronization of the active ingredient into fine particles via mechanical milling contributes to improvement of solubility, due to a large specific surface area of the particles, consequently resulting in improved dissolution rate and bioabsorption rate of the active drug.

The spray drying is a method of making fine particles by dissolving the active ingredient in a certain solvent and the spray-drying the resulting solution. During the spray-drying process, a high percent of the crystallinity of the phenanthrenequinone-based compound is lost to thereby result in an amorphous state, and therefore the spray-dried product in the form of a fine powder is obtained.

The mechanical milling is a method of grinding the active ingredient into fine particles by applying strong physical force to active ingredient particles. The mechanical milling may be carried out by using a variety of milling processes such as jet milling, ball milling, vibration milling, hammer milling, and the like. Particularly preferred is jet milling which can be carried out using an air pressure, at a temperature of less than 40° C.

Meanwhile, irrespective of the crystalline structure, a decreasing particle diameter of the particulate active ingredient leads to an increasing specific surface area, thereby increasing the dissolution rate and solubility. However, an excessively small particle diameter makes it difficult to prepare fine particles having such a size and also brings about agglomeration or aggregation of particles which may result in deterioration of the solubility. Therefore, in one preferred embodiment, the particle diameter of the active ingredient may be in a range of 5 nm to 500 μm. In this range, the particle agglomeration or aggregation can be maximally inhibited, and the dissolution rate and solubility can be maximized due to a high specific surface area of the particles.

Preferably, a surfactant may be additionally added to prevent the particle agglomeration or aggregation which may occur during formation of the fine particles, and/or an antistatic agent may be additionally added to prevent the occurrence of static electricity.

If necessary, a moisture-absorbent material may be further added during the milling process. The phenanthrenequinone-based compound of Formula 1 has a tendency to be crystallized by water, so incorporation of the moisture-absorbent material inhibits recrystallization of the phenanthrenequinone-based compound over time and enables maintenance of increased solubility of compound particles due to micronization. Further, the moisture-absorbent material serves to suppress coagulation and aggregation of the pharmaceutical composition while not adversely affecting therapeutic effects of the active ingredient.

Examples of the surfactant may include, but are not limited to, anionc surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate. These materials may be used alone or in any combination thereof.

Examples of the moisture-absorbent material may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Some of the above-mentioned moisture absorbents may also be used as the antistatic agent.

The surfactant, antistatic agent, and moisture absorbent are added in a certain amount that is capable of achieving the above-mentioned effects, and such an amount may be appropriately adjusted depending upon micronization conditions. Preferably, the additives may be used in a range of 0.05 to 20% by weight, based on the total weight of the active ingredient.

In one preferred embodiment, during formulation of the pharmaceutical composition according to the present invention into preparations for oral administration, water-soluble polymers, solubilizers and disintegration-promoting agents may be further added. Preferably, formulation of the composition into a desired dosage form may be made by mixing the additives and the particulate active ingredient in a solvent and spray-drying the mixture.

The water-soluble polymer is of help to prevent aggregation of the particulate active ingredients, by rendering surroundings of phenanthrenequinone-based compound molecules or particles hydrophilic to consequently enhance water solubility, and preferably to maintain the amorphous state of the active ingredient phenanthrenequinone-based compound.

Preferably, the water-soluble polymer is a pH-independent polymer, and can bring about crystallinity loss and enhanced hydrophilicity of the active ingredient, even under the between- and within-individual variation of the gastrointestinal pH.

Preferred examples of the water-soluble polymers may include at least one selected from the group consisting of cellulose derivatives such as methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose; polyvinyl alcohols; polyvinyl acetate, polyvinyl acetate phthalate, polyvinylpyrrolidone (PVP), and polymers containing the same; polyalkene oxide or polyalkene glycol, and polymers containing the same. Preferred is hydroxypropylmethyl cellulose.

In the pharmaceutical composition of the present invention, an excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. Accordingly, the solubilizer is preferably added to maximize the solubility of the formulation by modifying physical properties of the phenanthrenequinone-based compound.

In this respect, the solubilizer serves to enhance solubilization and wettability of the sparingly-soluble phenanthrenequinone-based compound, and can significantly reduce the bioavailability variation of the phenanthrenequinone-based compound originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug.

Preferred examples of the disintegration-promoting agent may include, but are not limited to, at least one selected from the group consisting of Croscarmellose sodium, Crospovidone, calcium carboxymethylcellulose, starch glycolate sodium and lower substituted hydroxypropyl cellulose. Preferred is Croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

The solvent for spray drying is a material exhibiting a high solubility without modification of physical properties thereof and easy volatility during the spray drying process. Preferred examples of such a solvent may include, but are not limited to, dichloromethane, chloroform, methanol, and ethanol. These materials may be used alone or in any combination thereof. Preferably, a content of solids in the spray solution is in a range of 5 to 50% by weight, based on the total weight of the spray solution.

The above-mentioned intestine-targeted formulation process may be preferably carried out for formulation particles prepared as above.

In one preferred embodiment, the oral pharmaceutical composition according to the present invention may be formulated by a process comprising the following steps:

(a) adding a phenanthrenequinone-based compound of Formula 1 alone or in combination with a surfactant and a moisture-absorbent material, and grinding the phenanthrenequinone-based compound of Formula 1 with a jet mill to prepare active ingredient microparticles;

(b) dissolving the active ingredient microparticles in conjunction with a water-soluble polymer, a solubilizer and a disintegration-promoting agent in a solvent and spray-drying the resulting solution to prepare formulation particles; and

(c) dissolving the formulation particles in conjunction with a pH-sensitive polymer and a plasticizer in a solvent and spray-drying the resulting solution to carry out intestine-targeted coating on the formulation particles.

The surfactant, moisture-absorbent material, water-soluble polymer, solubilizer and disintegration-promoting agent are as defined above. The plasticizer is an additive added to prevent hardening of the coating, and may include, for example polymers such as polyethylene glycol.

Alternatively, formulation of the active ingredient may be carried out by sequential or concurrent spraying of vehicles of Step (b) and intestine-targeted coating materials of Step (c) onto jet-milled active ingredient particles of Step (a) as a seed.

The oral pharmaceutical composition suitable for use in the present invention contains the active ingredient in an amount effective to achieve its intended purpose, that is therapeutic purpose. More specifically, a therapeutically effective amount refers to an amount of the compound effective to prevent, alleviate or ameliorate symptoms of disease. Determination of the therapeutically effective amount is well within the capability of those skilled in the art, especially in light of the detailed disclosure provided herein.

Further, the oral pharmaceutical composition according to the present invention is particularly effective for the treatment and/or prevention of metabolic diseases, degenerative diseases, and mitochondrial dysfunction-related diseases. Examples of the metabolic diseases may include, but are not limited to, obesity, obesity complications, liver diseases, arteriosclerosis, cerebral apoplexy, myocardial infarction, cardiovascular diseases, ischemic diseases, diabetes, diabetes-related complications and inflammatory diseases.

Complications caused from obesity include, for example hypertension, myocardiac infarction, varicosis, pulmonary embolism, coronary artery diseases, cerebral hemorrhage, senile dementia, Parkinson's disease, type 2 diabetes, hyperlipidemia, cerebral apoplexy, various cancers (such as uterine cancer, breast cancer, prostate cancer, colon cancer and the like), heart diseases, gall bladder diseases, sleep apnea syndrome, arthritis, infertility, venous ulcer, sudden death, fatty liver, hypertrophic cardiomyopathy (HCM), thromboembolism, esophagitis, abdominal wall hernia (Ventral Hernia), urinary incontinence, cardiovascular diseases, endocrine diseases and the like.

Diabetic complications include, for example hyperlipidemia, hypertension, retinopathy, renal insufficiency, and the like.

Examples of the degenerative diseases may include Alzheimer's disease, Parkinson's disease and Huntington's disease.

Diseases arising from mitochondrial dysfunction may include for example, multiple sclerosis, encephalomyelitis, cerebral radiculitis, peripheral neuropathy, Reye's syndrome, Friedrich's ataxia, Alpers syndrome, MELAS, migraine, psychosis, depression, seizure and dementia, paralytic episode, optic atrophy, optic neuropathy, retinitis pigmentosa, cataract, hyperaldosteronemia, hypoparathyroidism, myopathy, amyotrophy, myoglobinuria, muscular hypotonia, myalgia, reduced exercise tolerance, renal tubulopathy, renal failure, hepatic failure, hepatic dysfunction, hepatomegaly, sideroblastic anemia (iron-deficiency anemia), neutropenia, thrombocytopenia, diarrhea, villous atrophy, multiple vomiting, dysphagia, constipation, sensorineural hearing loss (SNHL), mental retardation, epilepsy, and the like.

As used herein, the term “treatment” refers to stopping or delaying of the disease progress, when the drug is used in the subject exhibiting symptoms of disease onset. The term “prevention” refers to stopping or delaying of symptoms of disease onset, when the drug is used in the subject exhibiting no symptoms of disease onset but having high risk of disease onset.

### DETAILED DESCRIPTION OF PREFERRED EMBODIMENTS

Now, the present invention will be described in more detail with reference to the following Examples. These examples are provided only for illustrating the present invention and should not be construed as limiting the scope and spirit of the present invention.

### Example 1

### Micronization of Active Ingredient Using Jet Mill

Micronizing of an active ingredient was carried out using a Jet mill (SJ-100, Nisshin, Japan). Operation was run at a supply pressure of 0.65 Mpa, and a feed rate of 16 to 20 g/hr 0.2 g of sodium lauryl sulfate (sodium lauryl sulfate) and 10 g of cryptotanshinone as a phenanthrenequinone-based compound were add to 100 ml of water and then ground for 10 hours. Micronized particles were recovered and a particle size was determined by zeta potential measurement. An average particle diameter was 1500 nm.

### Example 2

### Preparation of Spray-Drying Product

Cryptotanshinone per se or cryptotanshinone which was micronized in Example 1 was added to methanol. Then, a salt such as sodium chloride, a saccharide such as white sugar or lactose, or a vehicle such as microcrystalline cellulose, monobasic calcium phosphate, starch or mannitol, a lubricant such as magnesium stearate, talc or glyceryl behenate, and a solubilizer such as Poloxamer were added thereto, followed by homogeneous dispersion to prepare a spray-drying solution which will be used for subsequent spray-drying.

### Experimental Example 1

### Dissolution of Spray-Dried Formulation

To the spray-dried product of Example 2 were added approximately an equal amount of a water-soluble polymer (hydroxypropylmethyl cellulose) relative to an active ingredient, and vehicles such as Croscaimellose sodium and light anhydrous silicic acid, and the mixture was formulated without causing interference of disintegration. A drug dissolution test was carried out in a buffer (pH 6.8). All the compositions exhibited drug dissolution of 90% or higher after 6 hours.

### Experimental Example 2

### Evaluation of Relative Bioavailability of Spray-Dried Formulations in which the Phenanthrenequinone-Based Compound is Contained

10 male Sprague-Dawley rats were fasted, and the relative bioavailability in animals was evaluated for various formulations. Specifically, evaluation of the relative bioavailability was made for a preparation where a cryptotanshinone was roughly ground and was added in conjunction with 2% by weight of sodium lauryl sulfate (SLS) to an aqueous solution (preparation prior to grinding of an active ingredient), a preparation where a cryptotanshinone was ground into microparticles with a Jet mill, and was added in conjunction with 2% by weight of SLS to an aqueous solution (preparation after grinding of an active ingredient), a preparation where a formulation composed of the spray-dried product of Example 2 and the vehicle of Experimental Example 1 was added to an aqueous solution (spray-dried preparation), and a preparation where a cryptotanshinone was ground into microparticles with a Jet mill, formulated using the vehicle of Experimental Example 1 and added to an aqueous solution (solid-dispersed preparation).

TABLE 2

| | Blood conc. (ng/mL): Fasted | | | |
|---|---|---|---|---|
| | Preparation | Preparation | | |
| | before | after | Spray-dried | Solid-dispersed |
| Time (hour) | grinding | grinding | preparation | preparation |
| 0 | 0.00 | 0.00 | 0.00 | 0.00 |
| 0.5 | 176.92 | 150.55 | 368.54 | 373.49 |
| 1 | 96.38 | 169.12 | 458.65 | 475.19 |
| 2 | 154.91 | 205.35 | 246.42 | 241.38 |
| 3 | 256.63 | 222.51 | 272.35 | 281.39 |
| 6 | 135.81 | 421.16 | 880.82 | 889.66 |
| 12 | 77.30 | 141.15 | 252.94 | 256.04 |
| 24 | 65.90 | 137.98 | 166.90 | 166.10 |
| Avg. Cmax | 256.63 | 421.16 | 880.82 | 889.66 |
| Avg. AUC | 2491.82 | 4695.78 | 8132.44 | 8215.08 |
| (last) | | | | |

As can be seen from the results of Table 2, the spray-dried formulation and the solid-dispersed formulation, which were added to an aqueous solution, exhibited an about 3-fold increase of the bioavailability in a fasted state, as compared to the comparative formulation containing the same amount of the active ingredient, particularly the formulation prior to grinding of the active ingredient.

### Example 3

### Preparation of Intestine-Targeted Formulation

The spray-dried formulation prepared in Experimental Example 1 was added to an ethanol solution containing about 20% by weight of Eudragit S-100 as a pH-sensitive polymer and about 2% by weight of PEG #6,000 as a plasticizer, and the mixture was then spray-dried to prepare an intestine-targeted formulation.

### Experimental Example 3

### Acid Resistance of Intestine-Targeted Formulation

The intestine-targeted formulation prepared in Example 3 was exposed to pH 1.2 and pH 6.8, respectively. After 6 hours, the intestine-targeted formulation was removed and washed, and a content of an active ingredient was analyzed by HPLC. An effective amount of the active ingredient was assessed as a measure of the acid resistance. The acid resistance exhibited a very excellent result of 90 to 100%, thus suggesting that the intestine-targeted formulation is chemically stable in the stomach or small intestine.

### Experimental Example 4

### Measurement of Drug-Dissolution Profiles

After the intestine-targeted formulation was exposed to acidic environment of pH 1.2, as in Experimental Example 3, the acidity was changed to a value of pH 6.8 under artificial environment. A residual amount of the dissolved active ingredient was measured by HPLC. The results thus obtained are given in Table 3 below.

TABLE 3

| Time (min.) | Dissolution (%) at pH 6.8 |
|---|---|
| 0 | 0.00 |
| 10 | 68.48 |
| 30 | 82.99 |
| 45 | 88.48 |
| 60 | 88.62 |
| 120 | 91.20 |
| 180 | 91.43 |
| 240 | 94.18 |

### Experimental Example 5

### Therapeutic Efficacy of Intestine-Targeted Formulation

400 mg/kg of an intestine-targeted formulation in terms of active ingredient content was administered to ob/ob mice once a day, and changes in the body weight (BW) of animals were examined.

10-week-old ob/ob male mice (Jackson Lab) as an obese mouse model of type 2 diabetes were purchased from Orient Co. (Kyungki-do, Korea) and were allowed to acclimate to a new environment of the breeding room for 10 days prior to experiments. Animals were fed a solid feed (P5053, Labdiet) as a laboratory animal feed. The ob/ob male mice were housed and allowed to acclimate to a new environment for 10 days, in a breeding room maintained at a temperature of 22±2° C., humidity of 55±5%, and a 12-h light/dark (L/D) cycle (light from 8:00 a.m. to 8:00 p.m.). According to a randomized blocks design, the thus-acclimated animals were randomly divided into four groups, each consisting of 7 animals: a control group with administration of sodium lauryl sulfate (10 mg/kg), a group with administration of simply finely-divided powder of a cryptotanshinone (400 mg/kg), a group with administration of a jet-milled cryptotanshinone, and a group with administration of an intestine-targeted formulation of a ground cryptotanshinone. Each group of animals was given perorally (PO) 400 mg/kg of samples. Animals were fed solid feed pellets and water ad libitum. The changes in the body weight of animals in each group were measured.

As an experimental result, it was confirmed that the control group with administration of sodium lauryl sulfate and the group with administration of simply finely-divided powder of a cryptotanshinone were increased in body weight, whereas the group with administration of a jet-milled cryptotanshinone and the group with administration of an intestine-targeted formulation decreased in body weight. Particularly, the group with administration of an intestine-targeted formulation exhibited more than two times loss of body weight as compared to the group with administration of cryptotanshinone. Accordingly, the group with administration of the intestine-targeted formulation exhibited the highest loss (%) of body weight, thus confirming that excellent bioavailability is obtained.

### INDUSTRIAL APPLICABILITY

As apparent from the above description, an oral pharmaceutical composition according to the present invention increases a bioabsorption rate and an in vivo retention time of an active ingredient to thereby improve pharmacokinetic properties of the drug. As a result, it is possible to achieve desired therapeutic effects by increasing the bioavailability of a certain phenanthrenequinone-based compound as the active ingredient.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An oral pharmaceutical composition wherein a phenanthrenequinone-based compound by Formula 1 below, or a pharmaceutically acceptable salt, prodrug, solvate or isomer thereof, as an active ingredient, is prepared into an intestine-targeted formulation: 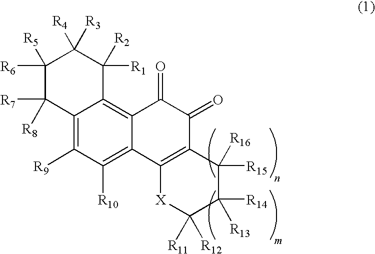
wherein
R₁, R₂, R₃, R₄, R₅, R₆, R₇, R₈, R₉, R₁₀, R₁₁, R₁₂, R₁₃, R₁₄, R₁₅ and R₁₆ are each independently hydrogen, halogen, hydroxyl or C₁-C₆ alkyl, alkene or alkoxy, C₄-C₁₀ cycloalkyl, heterocycloalkyl, aryl or heteroaryl, or two substituents thereof may be taken together to form a cyclic structure or form a double bond;
X is selected from the group consisting of C(R)(R′), N(R″), O and S, wherein R, R′ and R″ are each independently hydrogen or C₁-C₆ lower alkyl; and
m and n each independently are 0 or 1, with proviso that when m or n is 0, carbon atoms adjacent to m or n form a cyclic structure via a direct bond.

2. The composition according to claim 1, wherein X is O.

3. The composition according to claim 1, wherein the prodrug is a compound represented by Formula Ia below: 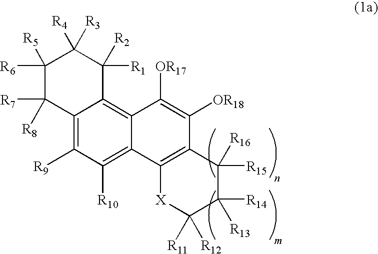
wherein,
R₁, R₂, R₃, R₄, R₅, R₆, R₇, R₃, R₉, R₁₀, R₁₁, R₁₂, R₁₃, R₁₄, R₁₅, R₁₆, m, n and X are as defined in Formula 1.
R₁₇ and R₁₈ are each independently —SO₃—Na⁺ or substituent represented by Formula 2 below or a salt thereof, 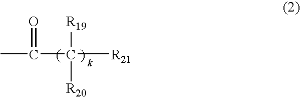
wherein,
R₁₉ and R₂₀ are each independently hydrogen or substituted or unsubstituted C₁˜C₂₀ linear alkyl or C₁˜C₂₀ branched alkyl
R₂₁ is selected from the group consisting of substituents i) to viii) below:
i) hydrogen;
ii) substituted or unsubstituted C₁˜C₂₀ linear alkyl or C₁˜C₂₀ branched alkyl;
iii) substituted or unsubstituted amine;
iv) substituted or unsubstituted C₃˜C₁₀ cycloalkyl or C₃˜C₁₀heterocycloalkyl;
v) substituted or unsubstituted C₄˜C₁₀ aryl or C₄˜C₁₀ heteroaryl;
vi) —(CRR′—NR″CO)₁—R₂₂, wherein R, R′ and R″ are each independently hydrogen or substituted or unsubstituted C₁˜C₂₀ linear alkyl or C₁˜C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃—Na⁺;
k is selected from the 0˜20, with proviso that when k is 0, R₁₉ and R₂₀ are not anything, and R₂₁ is directly bond to a carbonyl group.

4. The composition according to claim 1, wherein the compound of Formula 1 is selected from compounds of Formulas 3 and 4 below: 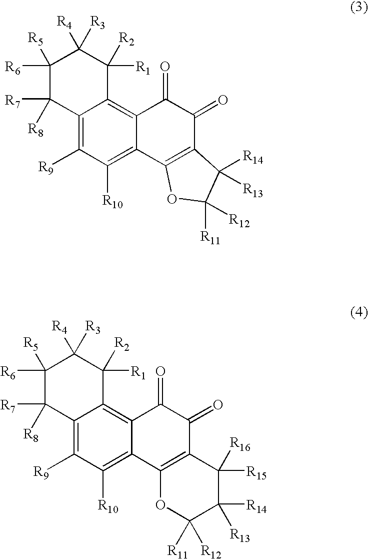
wherein, R₁, R₂, R₃, R₄, R₅, R₆, R₇, R₈, R₉, R₁₀, R₁₁, R₁₂, R₁₃, R₁₄, R₁₅ and R₁₆ are as defined in Formula 1.

5. The composition according to claim 1, wherein the compound of Formula 1 is selected from compounds of Formulas 5 and 6 below: 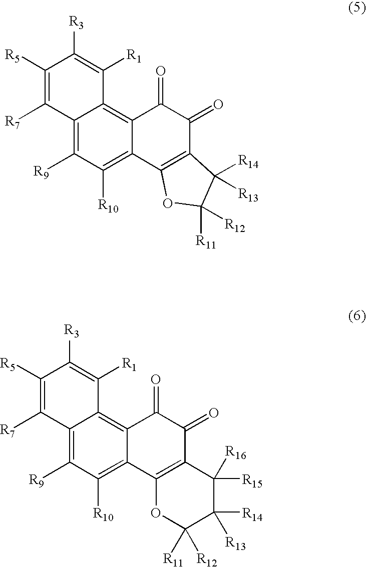
wherein, R₁, R₃, R₅, R₇, R₉, R₁₀, R₁₁, R₁₂, R₁₃, R₁₄, R₁₅ and R₁₆ are as defined in Formula 1.

6. The composition according to claim 1, wherein the compound of Formula 1 is a one of compounds below:

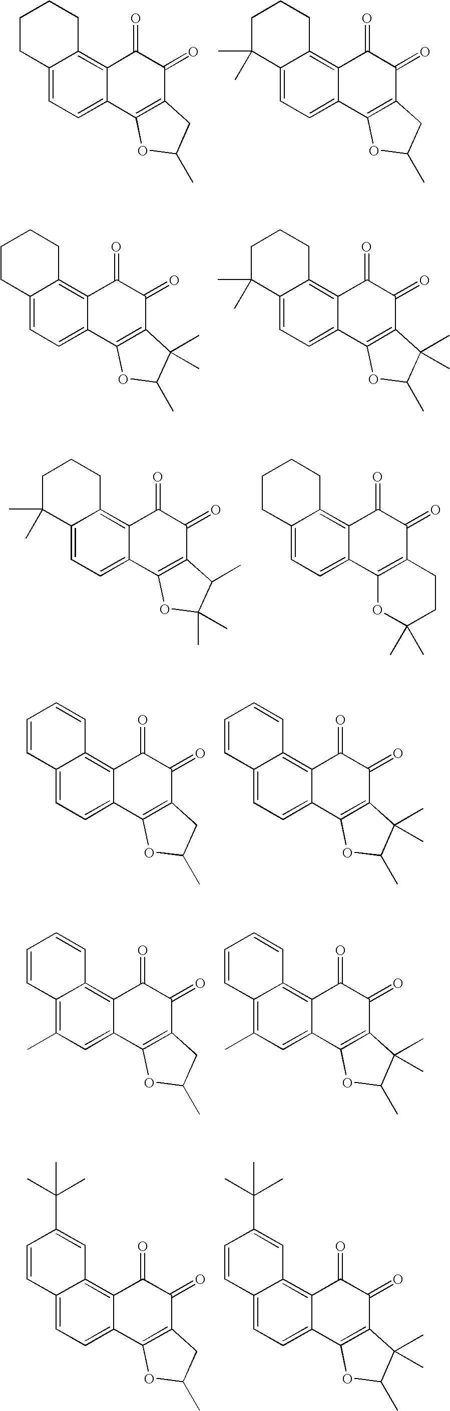

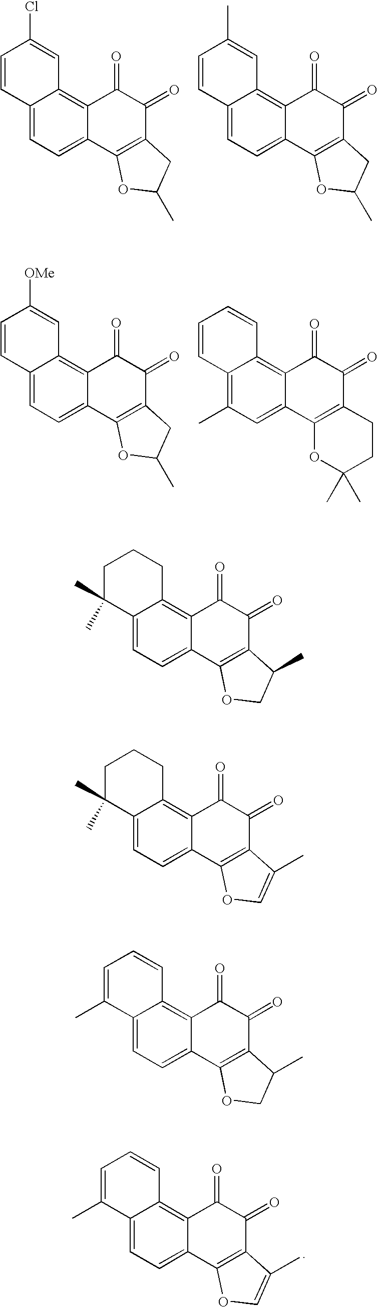

7. The composition according to claim 1, wherein the intestine-targeted formulation is carried out by addition of a pH sensitive polymer.

8. The composition according to claim 7, wherein the pH sensitive polymer is one or more selected from the group consisting of methacrylic acid-ethyl acrylate copolymer (Eudragit: Registered Trademark of Rohm Pharma GmbH), hydroxypropylmethyl cellulose phthalate (HPMCP), and a mixture thereof.

9. The composition according to claim 7, wherein the pH sensitive polymer is added by a coating process.

10. The composition according to claim 1, wherein the intestine-targeted formulation is carried out by addition of a biodegradable polymer which is decomposable by an intestine-specific bacterial enzyme.

11. The composition according to claim 10, wherein the polymer contains an azoaromatic linkage.

12. The composition according to claim 11, wherein the polymer containing the azoaromatic linkage is a copolymer of styrene and hydroxyethylmethacrylate (HEMA).

13. The composition according to claim 10, wherein the polymer is a naturally-occurring polysaccharide or a substituted derivative thereof.

14. The composition according to claim 13, wherein the polysaccharide or substituted derivative thereof is one or more selected from the group consisting of dextran ester, pectin, amylase and ethylcellulose or pharmaceutically acceptable salt thereof.

15. The composition according to claim 1, wherein the intestine-targeted formulation is carried out by addition of a biodegradable matrix which is decomposable by an intestine-specific bacterial enzyme.

16. The composition according to claim 15, wherein the matrix is a synthetic hydrogel based on N-substituted acrylamide.

17. The composition according to claim 1, wherein the intestine-targeted formulation is carried out by a configuration with time-course release of the drug after a lag time (‘time-specific delayed-release formulation’).

18. The composition according to claim 17, wherein the time-specific delayed-release formulation is carried out by addition of a hydrogel.

19. The composition according to claim 1, wherein the active ingredient have a crystalline structure.

20. The composition according to claim 1, wherein the active ingredient have a crystalline structure in which the crystallinity degree is 50% or less.

21. The composition according to claim 20, wherein the active ingredient have an amorphous structure.

22. The composition according to claim 1, wherein the active ingredient is contained in the form of a fine particle.

23. The composition according to claim 22, wherein the fine particles have particle diameters within a range of 5 nm to 500 μm

24. The composition according to claim 22, wherein the fine particles are prepared by spray drying of active material or mechanical milling.

25. The composition according to claim 24, wherein the mechanical milling is carried out by jet milling.

26. The composition according to claim 22, wherein one or more selected from the group consisting of surfactant, antistatic agent and moisture-absorbent is added during formation of the fine particles.

27. The composition according to claim 26, wherein the surfactant is one or more selected from the group consisting of anionic surfactants of docusate sodium and sodium lauryl sulfate; cationic surfactants of benzalkonium chloride, benzethonium chloride and cetrimide; nonionic surfactants of glyceryl monooleate, polyoxyethylene sorbitan fatty acid ester and sorbitan ester; amphiphilic polymers of polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate.

28. The composition according to claim 26, wherein the moisture-absorbent is one or more selected from the group consisting of colloidal silica, light anhydrous silicic acid, heavy anhydrous silicic acid, sodium chloride, calcium silicate, potassium aluminosilicate, and calcium aluminosilicate.

29. The composition according to claim 1, wherein during preparation of the formulation for oral administration, a water-soluble polymer, solubilizer and disintegration-promoting agent are added.

30. The composition according to claim 29, wherein the formulation is made by mixing additives and the active ingredient in the form of a fine particle in a solvent and then spray-drying the resulting mixture.

31. The composition according to claim 29, wherein the water-soluble polymer is one or more selected from the group consisting of methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose.

32. The composition according to claim 31, wherein the water-soluble polymer is hydroxypropylmethyl cellulose.

33. The composition according to claim 29, wherein the disintegration-promoting agent is one or more selected from the group consisting of Croscarmellose sodium, Crospovidone, calcium carboxymethylcellulose, starch glycolate sodium and lower substituted hydroxypropyl cellulose.

34. The composition according to claim 33, wherein the disintegration-promoting agent is Croscarmellose sodium.

35. The composition according to claim 29, wherein the solubilizer is a surfactant or amphiphile.

36. The composition according to claim 29, wherein 10 to 1000 parts by weight of the water-soluble polymer, 1 to 30 parts by weight of the disintegration-promoting agent and 0.1 to 20 parts by weight of the solubilizer are added based on 100 parts by weight of the active ingredient.

37. The composition according to claim 1, wherein the intestine-targeted formulation is prepared by a process comprising the following steps:
(a) adding a phenanthrenequinone-based compound of Formula 1 alone or in combination with a surfactant and a moisture-absorbent material, and grinding the phenanthrenequinone-based compound of Formula 1 with a jet mill to prepare active ingredient microparticles;
(b) dissolving the active ingredient microparticles in conjunction with a water-soluble polymer, a solubilizer and a disintegration-promoting agent in a solvent and spray-drying the resulting solution to prepare formulation particles; and
(c) dissolving the formulation particles in conjunction with a pH-sensitive polymer and a plasticizer in a solvent and spray-drying the resulting solution to carry out intestine-targeted coating on the formulation particles.

38. The composition according to claim 1, wherein the active ingredient exerts therapeutic effects for the treatment and/or prevention of metabolic diseases, degenerative diseases, and mitochondrial dysfunction-related diseases.
